(19) United States
(12) Patent Application Publication
(10) Pub. No.: US 2013/0090619 A1
(43) Pub. Date: Apr. 11, 2013

(54) DISPOSABLE ABSORBENT PRODUCT WITH MULTIPLE FLUID STORAGE STRUCTURES AND RELATED METHODS

(71) Applicants: Raquel CARBONARI, Philadelphia, PA (US); Thomas Bergin, Conshohocken, PA (US); Jehann Biggs, Evanston, IL (US); Lauren Entrekin, Upper Darby, PA (US)

(72) Inventors: Raquel CARBONARI, Philadelphia, PA (US); Thomas Bergin, Conshohocken, PA (US); Jehann Biggs, Evanston, IL (US); Lauren Entrekin, Upper Darby, PA (US)

(73) Assignee: SCAHYGIENE PRODUCTS AB, Goteborg (SE)

(21) Appl. No.: 13/269,292

(22) Filed: Oct. 7, 2011

(51) Int. Cl. A61F 13/51; B23P 11/00

(52) U.S. Cl. 604/378; 29/428

(57) ABSTRACT

A disposable absorbent product includes a backsheet, a topsheet overlaying the backsheet, and a core disposed between the backsheet and topsheet for retaining fluid secreted by a wearer of the absorbent product. The core has a length dimension, a width dimension, and a thickness dimension orthogonal to the length and width dimensions. The core includes first and second fluid storage structures, with the first fluid storage structure at least partially surrounding the second fluid storage structure in the thickness dimension.

### TECHNICAL FIELD

The present invention is generally related to absorbent products and, more particularly, to disposable absorbent products worn by humans for the containment and absorption of fluid bodily secretions.

### BACKGROUND

Disposable absorbent products for absorption of bodily fluids are available in different types, designs, and dimensions. For example, baby diapers, adult diapers, and incontinence guards are products designed for the containment of urine and excrement. There are other types of disposable absorbent articles, such as feminine hygiene products (e.g., heavy and light incontinence pads, pantiliners) that are designed to contain and absorb urine and/or menses secreted by female wearers. Known products of this type typically include a topsheet facing the body of the wearer, a backsheet facing the garment worn by the wearer, and an absorbent core sandwiched between the topsheet and backsheet.

In conventional products of that type, the core may include a pair of discrete fluid storage structures, for example stacked over one another. U.S. Pat. No. 5,855,572 illustrates that type of design. This type of stacked arrangement, however, may result in products that are relatively bulky and therefore unappealing and/or uncomfortable to the wearer.

Other known products have addressed the above-discussed bulkiness by having a core in which one of the discrete fluid storage structures is a relatively thin layer of absorbent material, such as an airlaid-based structure or a foam structure. A problem with some of these thin layers of material is their rigidity, and more specifically the rigidity along the edges and at the corners of those layers. This rigidity may cause discomfort to the wearer. And while rounded/chamfered corners and/or edges may address the problems associated with the rigidity of these materials, the required rounding/chamfering increases the overall cost and complexity of the manufacturing processes involved. Moreover, rounding/chamfering may result in the production of trimmed portions of material, which further increases material costs, and may require additional equipment and processes for recycling or disposition of those trimmed portions. Additionally, the rounding/chamfering of fluid storage structures of this type reduces the overall absorption capacity of the products of which those structures form part, by virtue of the removal of those portions of material otherwise available for storing fluid.

It would be advantageous, therefore, to provide disposable absorbent products, and related methods, that address these and other shortcomings of conventional disposable absorbent products of the type described above.

### BRIEF DESCRIPTION OF THE DRAWINGS

The objectives and features of the invention will become more readily apparent from the following detailed description taken in conjunction with the accompanying drawings in which:

FIG. 1 is a perspective view of a disposable absorbent product in accordance with one embodiment of the invention.

FIG. 2 is a partially disassembled view of the disposable absorbent product of FIG. 1.

FIG. 3 is a perspective view of a core of the disposable absorbent product of FIGS. 1 and 2.

FIG. 4 shows lateral and frontal elevation views of the core of FIG. 3.

FIG. 5 is a top view of the core of FIGS. 3 and 4.

FIG. 6 is a frontal elevation view of the core of FIGS. 3-5 during use.

FIG. 7 is a top view, similar to FIG. 5, illustrating a core in accordance with another embodiment of the invention.

FIG. 8 is a top view, similar to FIGS. 5 and 7, illustrating a core in accordance with yet another embodiment of the invention.

FIG. 9 is a partially disassembled view of a core in accordance with another embodiment of the invention.

FIG. 10 is an assembled view of the core of FIG. 9.

FIG. 11 is a schematic view of an apparatus for forming a portion of the core of FIGS. 9 and 10, in accordance with an embodiment of the invention.

FIG. 11A is a schematic view of an apparatus for forming a portion of the core of FIGS. 9 and 10, in accordance with another embodiment of the invention.

FIG. 12 is a schematic view of an apparatus and process for forming a portion of the core of FIGS. 9 and 10, in accordance with yet another embodiment of the invention.

### SUMMARY

In one embodiment, a disposable absorbent product is provided. The absorbent product includes a backsheet, a topsheet overlaying the backsheet, and a core disposed between the backsheet and topsheet for retaining fluid secreted by a wearer of the absorbent product. The core has a length dimension, a width dimension, and a thickness dimension orthogonal to the length and width dimensions. The core includes first and second fluid storage structures, with the first fluid storage structure at least partially surrounding the second fluid storage structure in the thickness dimension.

In a specific embodiment, the first fluid storage structure includes a top surface adjacent the topsheet, a bottom surface adjacent the backsheet, and a hole extending from the top surface to the bottom surface, with the second fluid storage structure being located within the hole. In another specific embodiment, the first fluid storage structure has an inner portion having a first thickness, and an outer portion having a second thickness that is greater than the first thickness and which surrounds the inner portion in the thickness dimension. In that embodiment, the second fluid storage structure is supported within the inner portion. Additionally, the first and second fluid storage structures may include respective first and second, generally coplanar top surfaces, both adjacent the topsheet.

In another specific embodiment, the second fluid storage is spaced from the first fluid storage structure at least along a portion of the periphery of the second fluid storage structure. The first fluid storage structure may have pulp. Additionally or alternatively, the second fluid storage structure may comprise an airlaid material. The second fluid storage structure may include, in specific embodiments, a superabsorbent material (“SAP”). In some embodiments, the second fluid storage structure has an absorption capacity of at least about 60 grams Rothwell.

In another embodiment, a disposable absorbent product is provided that includes a backsheet, a topsheet overlaying the backsheet, and a core disposed between the backsheet and topsheet for retaining fluid secreted by a wearer of the disposable absorbent product. The core has a length dimension, a width dimension, and a thickness dimension orthogonal to the length and width dimensions. Further, the core includes a first fluid storage structure having pulp, and a second fluid storage structure that has an airlaid material. The first fluid storage structure at least partially surrounds the second fluid storage structure in the thickness dimension.

In another embodiment, an absorbent core is provided for use in an absorbent product. The absorbent core comprises a first fluid storage structure and a second fluid storage structure. The core has a length dimension, a width dimension, and a thickness dimension orthogonal to the length and width dimensions. The first fluid storage structure at least partially surrounds the second fluid storage structure in the thickness dimension.

In yet another embodiment, a method is provided for forming a core in a disposable absorbent product that includes a topsheet, a backsheet overlaying the topsheet, and a core disposed between the topsheet and backsheet and which has a length dimension, a width dimension, and a thickness dimension orthogonal to the length and width dimensions. The method includes obtaining a first fluid storage structure having a top surface, a bottom surface opposite the top surface, a plurality of outer walls extending between the top and bottom surfaces, and a plurality of inner walls. The outer and inner walls substantially extend along dimensions orthogonal to the thickness dimension, and the inner walls define an opening in the first fluid storage structure. The method includes locating a second fluid storage structure within the opening, and placing the first and second fluid storage structures between the topsheet and backsheet to thereby define the core of the disposable absorbent product.

### DETAILED DESCRIPTION

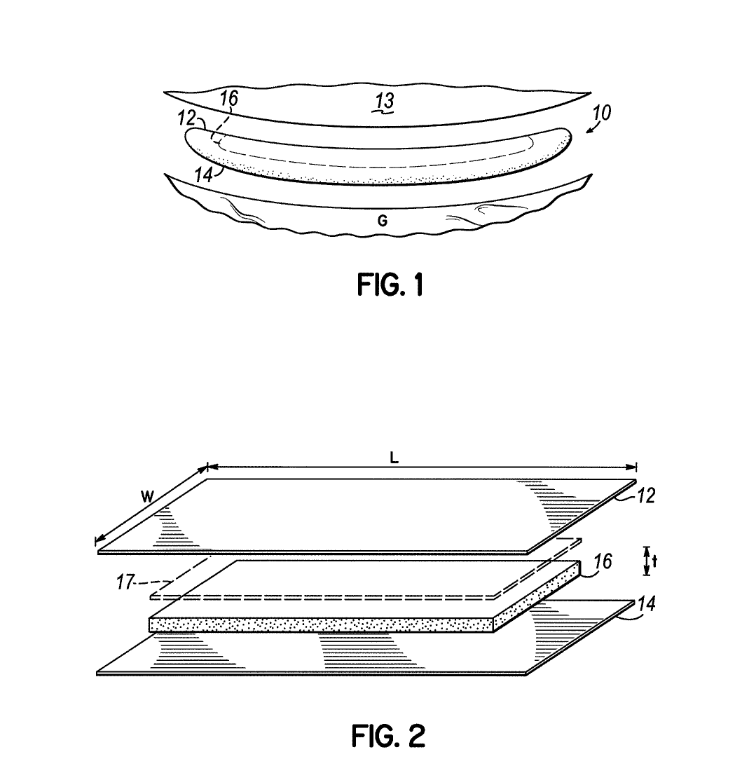

With reference to the figures, and more particularly to FIG. 1, an exemplary disposable absorbent product in the form of a feminine pad 10 includes a topsheet 12, and a backsheet 14 disposed opposite the topsheet 12. When worn, the topsheet 12 faces the body of the wearer, schematically represented and assigned the numeral 13, while the backsheet 14 faces away from the body 13 of the wearer. In the case of known uses of feminine pads and similar products, the backsheet 14 faces a garment G worn by the wearer. While not shown, the feminine pad 10 may include one or more features such as lateral extensions resembling wings, adhesive components, or mechanical entanglement-type (hook-and-loop) fasteners that allow the wearer to secure the pad 10 to the garment G. Additionally or alternatively, and while also not shown, the pad 10 may include adhesive or mechanical components that allow the pad 10 to be secured directly onto the body 13 of the wearer. The pad 10 also includes a core, generally assigned the numeral 16, that is configured to absorb and retain body fluids, such as urine and/or menses, secreted by the wearer.

With continued reference to FIG. 1, and further referring to FIGS. 2-4, the core 16 and the pad 10 of which core 16 forms part, extend along a length dimension L, a width dimension W, and a thickness dimension t orthogonal to the length and width dimensions L, W. Further, the core 16 has a generally rectangular outer profile in the plane defined by the length and width dimensions L, W (the “product plane”), although those of ordinary skill in the art will readily appreciate that such shape is exemplary rather than limiting. For example, and without limitation, the core 16 may instead have a generally hourglass shape, or some other regular or irregular shape, symmetrical or asymmetrical. The core 16 may optionally include an acquisition layer 17 (FIG. 2) or similar structure that is primarily designed to acquire and/or distribute fluids received through the topsheet 12 and direct same toward other portions of the core 16 that are primarily designed to store the fluid, as explained in further detailed below. In that regard, it is contemplated that the optional acquisition layer 17 may, in certain embodiments, be free of fluid-storage materials such as superabsorbent material (“SAP”) and/or be free of fluff pulp.

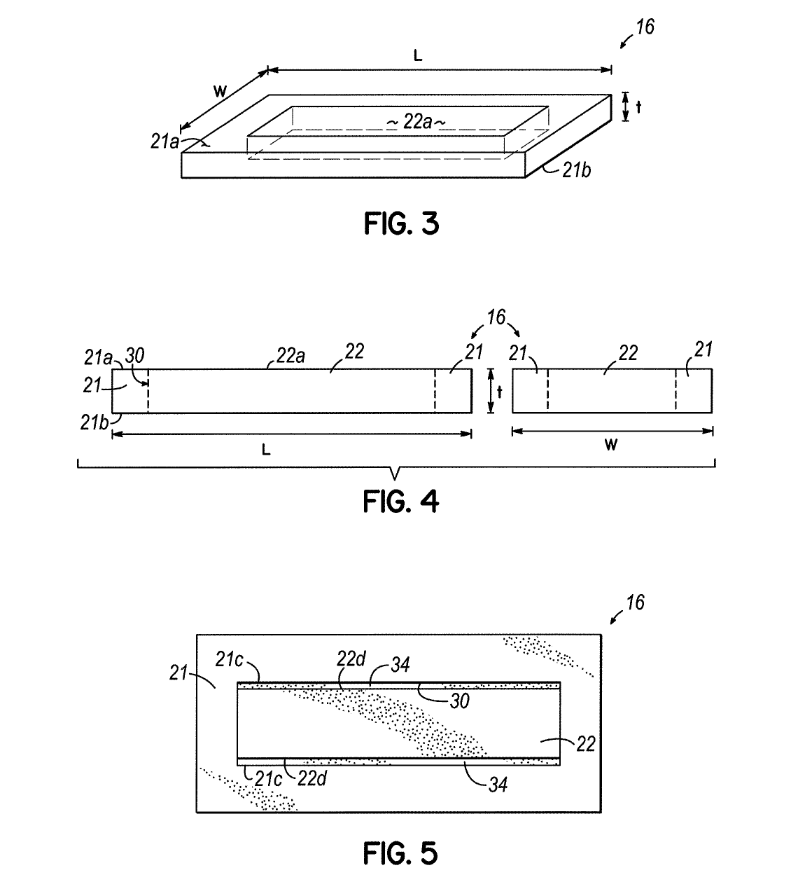

Referring particularly to FIGS. 3 and 4, the core 16 of that exemplary embodiment includes two discrete fluid storage structures, although the principles disclosed herein are similarly applicable to cores having more than two discrete fluid storage structures. In the illustrative embodiment of FIGS. 3 and 4, a first fluid storage structure 21 and a second fluid storage structure 22 are arranged in the core 16 such that the first fluid storage structure 21 surrounds the second fluid storage structure 22 in the thickness dimension t, as illustrated in those figures. More specifically in that embodiment, the second fluid storage structure 22 rests within a centrally located hole or opening 30 that extends through the entire thickness of the first fluid storage structure 21 i.e., between the top and bottom surfaces 21a, 21b of the first fluid storage structure 21. While not shown, the core 16 may also include other components such as non-woven or paper-based materials (e.g., tissue) that at least partially wrap the first and second fluid storage structures 21, 22.

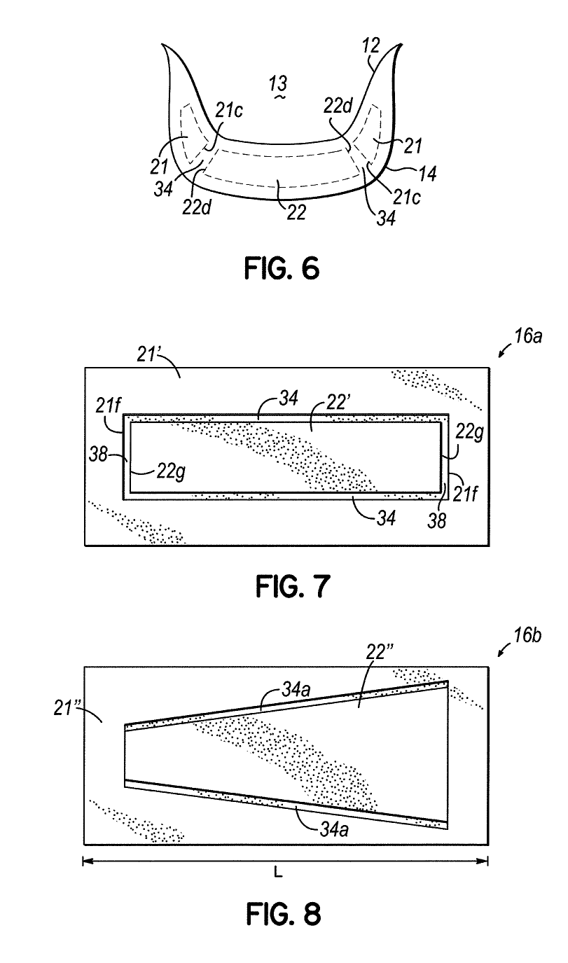

Referring further now to FIGS. 5-8, the size and shape of the first and second fluid storage structures 21, 22 may be chosen such that the second fluid storage structure 22 is spaced from the first fluid storage structure 21 at least along a portion of the perimeter of the second fluid storage structure 22. Accordingly, in the exemplary embodiment shown in FIG. 5, the inner lateral walls 21c of the first fluid storage structure 21 are spaced from the lateral walls 22d of the second fluid storage structure 22. The spacing between the lateral walls 21c and 22d defines a pair of lateral channels 34 that permit the pad 10 to take-on a “cup” shape (FIG. 6) when worn, thus allowing the core 16 and the rest of the pad 10 to generally conform to the body 13 of the wearer. The “cup” shape (FIG. 6) attained by the core 16 facilitates the containment of bodily fluid secreted by the wearer and directs the body fluid trapped in the channels 34 to enter the first and second fluid storage structures 21, 22 through the lateral walls 21c, 22d. A contemplated variation of core 16 is illustrated in FIG. 7, in which like reference numerals refer to similar features in FIGS. 1-6. In that alternative embodiment, the core 16a shown in that figure has first and second fluid storage structures 21′, 22′, in which the second fluid storage structure 22′ is spaced from the first fluid storage structure 21′ along the entire periphery of the second fluid storage structure 22′. In that regard, the inner end walls 21f of the first fluid storage structure 21′ are spaced from the respectively confronting end walls 22g of the second fluid storage structure 22′. The spacing between the end walls 21f and 22g define a pair of end channels 38, having functionality similar to that of the lateral channels 34 described above.

While FIGS. 5-7 depict embodiments in which both lateral walls 22d and/or both end walls 22g of the second fluid storage structure 22, 22′ are spaced from the respectively confronting inner walls 21c, 21f of the first fluid storage structure 21, 21′, other alternatives are contemplated that fall within the scope of the present disclosure. For example, and without limitation, only one of the lateral walls 22d and/or only one of the end walls 22g of the second fluid storage structure 22, 22′ may be spaced from the corresponding confronting inner wall 21c or 21f of the first fluid storage structure 21, 21′, while the other of the lateral walls 22d or of the end walls 22g may be substantially adjacent the respectively confronting inner wall 21c or 21f. Accordingly, while FIGS. 5-7 depict embodiments in which the channels 34, 38 define substantially symmetrical cores 16, 16a, it is contemplated that they may instead define asymmetrical cores, and still fall within the scope of the present disclosure.

In addition to the above, those of ordinary skill in the art will readily appreciate that the channels 34, 38 may or may not extend substantially along the length and width dimensions L, W, but be instead oriented so as to extend in any direction, depending on the shapes of the first and second fluid storage structures 21, 21′ 22, 22′. FIG. 8 illustrates that type of contemplated alternative embodiment. In that embodiment, two lateral channels 34a are defined between first and second fluid storage structures 21″ and 22″ of the core 16b shown in that figure. The lateral channels 34a extend along directions that are transverse, rather than parallel, to the length dimension L of the core 16b.

Referring again to FIGS. 3 and 4, the respective thicknesses of the first and second fluid storage structures 21, 22 in those embodiments are substantially equal. In that regard, the top surfaces 21a, 22a of the first and second fluid storage structures 21, 22, adjacent the topsheet 12, are substantially coplanar i.e., the top surfaces 21a, 22a lie in planes that are spaced from one another by no more than about 5 mm. In those embodiments, accordingly, the resulting core 16 has a uniform thickness, which enhances the wearer's comfort and may be perceived as more aesthetically pleasing than cores having a non-uniform thickness. Further, this type of core has the advantages gained by having more than one (e.g., two) discrete fluid storage structures, without the need to increase the overall thickness of the core 16.

In one specific embodiment, the first fluid storage structure 21 has natural or synthetic fluff (e.g, cellulose fluff pulp), while the second fluid storage structure 22 is a generally rectangular structure (i.e., in a plane orthogonal to the thickness dimension t) made of a relatively rigid airlaid material which may or may not contain fluff pulp. In that embodiment, one or both of the fluid storage structures 21 and 22 may contain SAP or some other fluid-storage material. As used herein, the term “fluid storage structure” is intended to describe absorbent structures, forming part of the core of a disposable absorbent product, that substantially has a fluid storage function. In that regard, and without limitation, the fluid storage structures contemplated to fall within the scope of the present disclosure may contain natural or synthetic materials (e.g., SAP) that are predominantly configured to store, rather than to acquire or distribute fluid to other components that are intended to ultimately store the fluid secreted by the wearer. For example, fluid storage structures of the type contemplated herein may have a storage capacity in the range of about 60 grams to about 6000 grams, as measured by the Rothwell method (ISO 11948-1), known to those of ordinary skill in the art, and the description of which falls beyond the scope of the present disclosure.

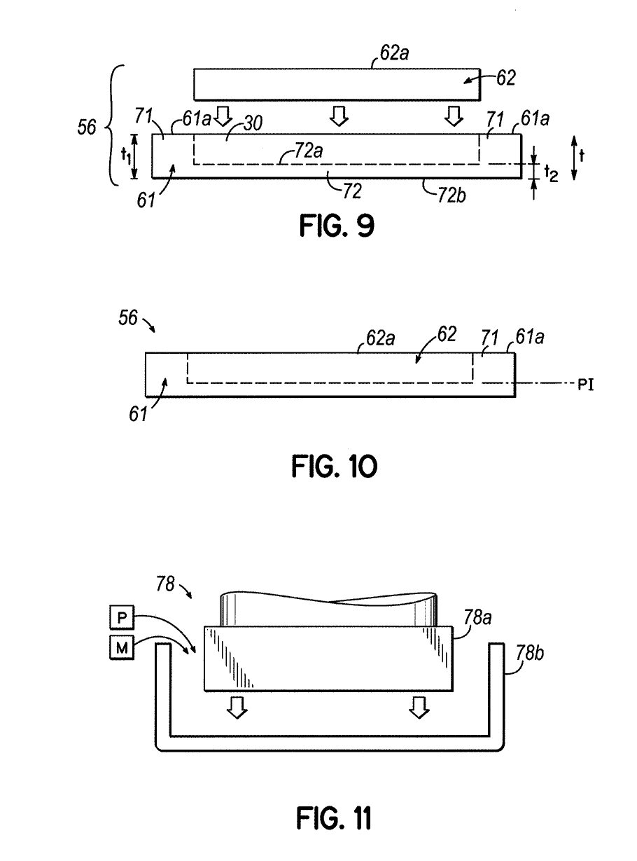

Referring now to FIGS. 9 and 10, in which like reference numerals refer to similar features in the preceding figures, another embodiment of a core 56 is illustrated. Core 56 includes discrete first and second fluid storage structures 61, 62, with the first fluid storage structure 61 surrounding the second fluid storage structure 62 in the thickness dimension t. FIG. 9 illustrates a thickness difference in the first fluid storage structure 61, between an outer region 71 and a relatively thinner inner region 72. More specifically, the outer region 71 has a thickness t₁ that is greater than the thickness t₂ of the inner region 72. In that regard, the second fluid storage structure 62 rests within the shallower inner region 72, and is supported on a top surface 72a of the inner region 72. The top surface 72a of inner region 72 extends in a plane PI that is intermediate between the bottom surface 72b of the first fluid storage structure 61 and the top surface 61a of the outer region 71. The thicknesses t₁ and t₂ are suitably chosen such that, when the core 56 is assembled (FIG. 10), a top surface 62a of the second fluid storage structure 62 is substantially coplanar with the top surface 61a of the first fluid storage structure 61 at the outer region 71 i.e., the top surfaces 61a, 62a lie in planes that are spaced from one another by no more than about 5 mm. This configuration results in a core 56 having a uniform thickness, with advantages similar to those described with respect to the core 16 of the preceding figures.

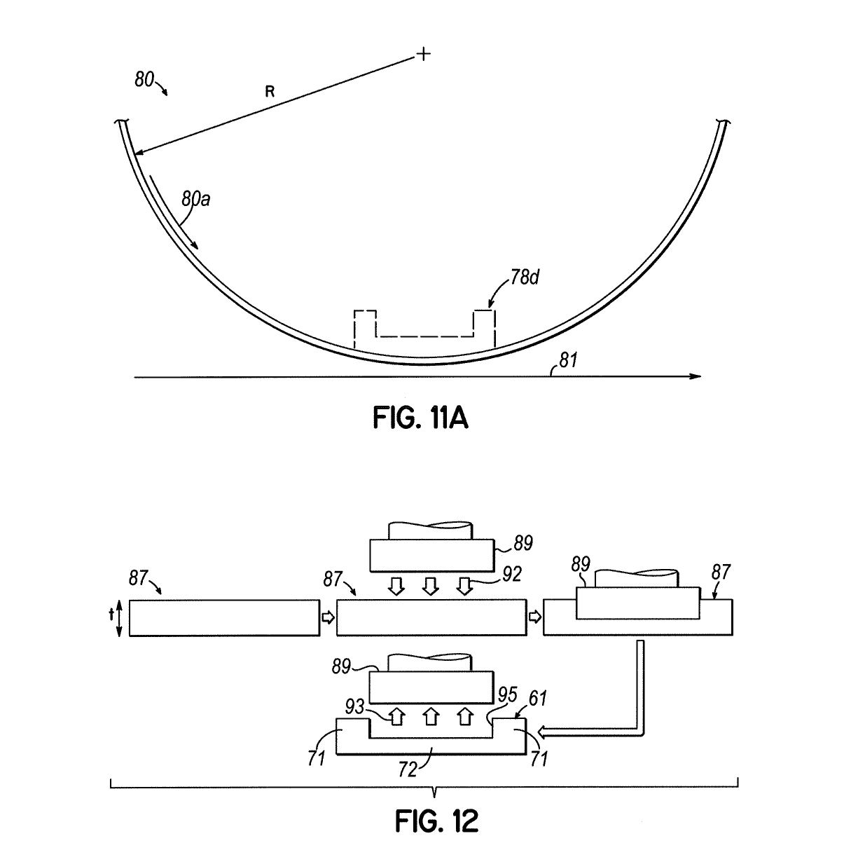

With continued reference to FIGS. 9-10, and further referring to FIGS. 11, 11A, and 12, the gradient in thickness between the inner and outer regions 72, 71 of the first fluid storage structure 61 may be achieved by at least one of two contemplated methods. A first exemplary method, schematically illustrated in FIG. 11, includes forming the first fluid storage structure 61 (FIGS. 9-10) in a mold 78, by directing pulp P and/or other materials, as well as materials M primarily having a fluid storage function (e.g., SAP) into the mold 78. The mold 78 has the desired shape of the first fluid storage structure 61 to be formed. The mold 78 may be a single-component structure or instead may, as illustrated in FIG. 11, have a male mold component 78a and a female mold component 78b. The formed first fluid storage structure 61 may be then removed from the mold 78 and advanced to another stage of the process (not shown) to receive the second fluid storage structure 62 within the formed centrally located opening 30 of the first fluid storage structure 61.

Alternatively, the components making up the second fluid storage structure 62 may be added to the mold 78 so as to be received within the opening 30 on the top surface 72a. To that end, when using the exemplary two-component mold 78 illustrated in FIG. 11, this part of the process would entail moving the male and female components 78a, 78b away from one another so that the material(s) making up the second fluid storage structure 62 can be placed into the opening 30. The assembled structure i.e., the assembly made up of the first fluid storage structure 61 and the second fluid storage structure 62 may then be removed from the mold 78 and advanced to another stage of the pad manufacturing process.

FIG. 11A illustrates a variation of the process described above, in which a mold 78d forms part of a rotating drum 80, which may include one or more molds 78d (only one shown). Mold 78d has the desired shape of the first fluid storage structure 61 to be formed. The core-forming process thus entails directing pulp P and/or other materials, as well as materials M primarily having a fluid storage function (e.g., SAP) into the mold 78d and incorporating the material(s) making up the second fluid storage structure 62 in a subsequent step.

As described above, the second fluid storage structure 62 may be in the form of a relatively rigid layer of material (e.g., an airlaid-based material). A contemplated process for making the core 56 includes cutting a continuous web defining the rigid layer of material with a cut-and-slip apparatus and process and then placing the discrete cut piece of the rigid layer of material into the opening 30 (FIG. 9). The above-referenced continuous web may be made “in-line” i.e., as another part of the process for making the core 56, on the same manufacturing line as that for manufacturing the rest of the core 56. Alternatively, the above-referenced continuous web may be made off-line and supplied to the manufacturing line as a prefabricated web.

An exemplary cut-and-slip apparatus and process is described in U.S. Pat. No. 6,544,375, entitled “Process for Applying Discrete Web Portions to a Receiving Web,” the contents of which are hereby expressly incorporated by reference herein. In this regard, embodiments of the invention in which the first fluid storage structure 61 is predominantly a relatively soft structure (e.g., made primarily from fluff pulp) and in which the second fluid storage structure 62 is a relatively rigid material, prevent exposure of the edges and/or corners of the second fluid storage structure 62. This, in turn, prevents or at least minimizes the likelihood of contact of those edges and/or corners with the wearer, which enhances the wearer's comfort. In addition, embodiments of this type facilitate the attainment of complex shapes (i.e., hourglass shape or some other regular or irregular shape) for the core 16, 56, while maintaining the simplicity in the processing of the relatively rigid second fluid storage structure 62. More specifically, embodiments are contemplated in which the first fluid storage structure 61 is a soft structure, made primarily of fluff pulp, and made, for example, in a drum mold having the desired regular or irregular shape of the storage structure 61. Further, in those embodiments, the relatively rigid material making up the second fluid storage structure 62 is cut into a simple shape e.g., a rectangular shape, using a simple process such as the cut-and-slip process discussed above. The end-result in those embodiments is a core 16, 56 having at least two fluid storage structures 61, 62, and also having the desired overall shape, absorption capacity, and high level of comfort for the wearer, without the drawbacks of conventional cores.

Yet another advantage of embodiments of the type described above, in which a relatively soft fluid storage structure surrounds a relatively rigid fluid storage structure, is the resulting enhanced fluid containment. More specifically, fluid is known to flow from an open, low density capillary structure, to a high density capillary structure, while it is known not to flow from a high density to a low-density capillary structure. In that regard, fluid received by the relatively high-density, rigid second fluid storage structure 62, will not have a tendency to flow outwardly, toward the relatively low-density, soft first fluid storage structure 61. Fluid received by the rigid second fluid storage structure 62, accordingly, will have a tendency to remain in that storage structure 62, which enhances the overall containment of fluid by the core 16, 56.

Whether the first fluid storage structure 61 and second fluid storage structure 62 are assembled together within a mold, or made at two different locations along the process, the assembled structure is then placed between a pair of confronting webs defining the topsheet 12 and backsheet 14 of the pad 10, for further disposition in the pad-manufacturing process. Prior to or subsequent to the placement of the assembled structure between the webs defining the topsheet 12 and backsheet 14, and while not shown, it is contemplated that the assembled structure may be calendered or passed through some other apparatus exerting an even amount of pressure over the first and second fluid storage structures 61, 62. Accordingly, in the embodiment illustrated at FIGS. 9 and 10, the core 56 therein may be made utilizing the method described above and result in a core in which the density of material (e.g., fluff pulp) in the outer region 71 is similar to that of the inner region 72.

While the process associated with FIG. 11 is described with reference to the core 56 of FIGS. 9 and 10, this is not intended to be limiting. Accordingly, it is contemplated that the principles discussed in connection with that process are similarly applicable to the formation of the core 16, 16a, or 16b of FIGS. 1-8.

FIG. 12 schematically illustrates another exemplary method for forming the core of pad 10. Specifically, that method may be used to form the first fluid storage structure 61 of the exemplary core 56 (FIGS. 9-10). That method includes forming the first fluid storage structure 61 so as to first obtain a partially-formed fluid storage structure 87 of generally uniform thickness. A schematically represented apparatus 89 (e.g., a calender roll) is then used to apply pressure (arrows 92) against a selected, central portion of the partially-formed fluid storage structure 87. That selected portion corresponds to the inner region 72 of the first fluid storage structure 61 (FIGS. 9-10). Movement of the apparatus 89 away from the first fluid storage structure 61 (arrows 93) leaves that structure 61 available to then receive the second fluid storage structure 62 (FIGS. 9-10) within a centrally located opening 95 formed in the first fluid storage structure 61. The localized pressure exerted by the apparatus 89 results in an inner region 72 that has a higher density of material (e.g., fluff pulp) than that of the outer region 71.

The exemplary apparatus and process described with reference to FIG. 12 contemplates leaving the outer region 71 of the first fluid storage structure 61 substantially uncompressed, with the pressure exerted by apparatus 89 being limited only to the portion corresponding to the inner region 72. A contemplated variant includes compressing both, the inner and outer regions of the partially-formed structure 87, but to different extents. More specifically, that alternative embodiment contemplates applying a first pressure to an outer region of the partially-formed structure 87, and a second pressure to an inner region of the partially-formed structure 87, with the second pressure being greater than the first pressure. Accordingly, in that embodiment, the inner region is compressed to a greater extent than the outer region, thereby resulting in the difference in thickness between the outer region 71 and inner region 72. The resulting density of fluff pulp for embodiments in which that type of material is substantially present in the first fluid storage structure 61, may be in the range from about 0.07 g/cm³ to about 0.3 g/cm³ in specific embodiments. Those of ordinary skill in the art will readily appreciate, however, that this range of densities is merely exemplary and thus not intended to be limiting.

Referring generally to the different embodiments illustrated in the preceding figures, the thicknesses of the first fluid storage structure 21, 21′, 21″, 61 and of the second fluid storage structure 22, 22′, 22″, 62 are suitably chosen, as are the basis weights of materials making up those two fluid storage structures. For example, and without limitation, the first fluid storage structure 21, 21′, 21″, 61 may include fluff pulp in a basis weight in the range from about 200 g/m² to about 600 g/m². In addition or alternatively, the first fluid storage structure 21, 21′, 21″, 61 may include SAP in a basis weight in the range from about 0 g/m² to about 600 g/m². The thickness of the first fluid storage structure 21, 21′, 21″, 61 may be in the range from about 0.5 mm to about 12 mm. In another non-limiting example, the second fluid storage structure 22, 22′, 22″, 62 may be made of an airlaid material and/or contain SAP. The airlaid material in that example may be present in a basis weight in the range from about 80 g/m² to about 1000 g/m², for example, while the SAP may be present in a weight-percentage in the range from about 5% to about 60%. In that example, moreover, the thickness of the second fluid storage structure 22, 22′, 22″, 62 may be in the range from about 0.5 mm to about 12 mm, for example. Exemplary airlaid materials suitable as the second fluid storage structure 22, 22′, 22″, 62 are materials known under the names “Airlaid 460 g/m2, 45% SAP, Multibonded, 4.0 mm;” “w76, 460 g/m², multibond material, fw1200;” and “w86, 600 g/m², multibond material, fw1000,” all commercially available from Glatfelter Falkenhagen GmbH, of Falkenhagen, Germany. In yet another example, the second fluid storage structure 22, 22′, 22″, 62 may be a foam-based material that may or may not contain SAP. Example of suitable foam-based materials are High Internal Phase Emulsion (HIPE) foams, such as those described in U.S. Pat. Nos. 5,387,207; 5,260,345; 5,650,222; and 5,849,805, the respective disclosures of which are hereby expressly incorporated by reference herein in their entirety.

Those of ordinary skill in the art will readily appreciate that, while the embodiments illustrated and described herein refer to a feminine pad, they are similarly applicable to other types of disposable absorbent products. For example, and without limitation, the principles and structures described herein are similarly applicable to baby diapers, adult diapers, incontinence guards, pantiliners, and other products in which absorbent structures are required for containment and absorption of fluids secreted by a wearer. Similarly, it is contemplated that the structures described herein can be used to manufacture only absorbent cores, rather than full disposable absorbent products. More specifically, embodiments are contemplated consisting of a core insert manufactured in one location and which is then supplied to another manufacturing location or to a consumer, to be used with a disposable absorbent product or even with a non-disposable absorbent product (e.g., underwear or brief).

Exemplary embodiments of the invention are described as follows, in non-limiting fashion:
1. A disposable absorbent product comprising:

a backsheet;

a topsheet overlaying said backsheet; and

a core disposed between said backsheet and said topsheet for retaining fluid secreted by a wearer of the absorbent product, said core having a length dimension, a width dimension, and a thickness dimension orthogonal to the length and width dimensions, said core including first and second fluid storage structures, said first fluid storage structure at least partially surrounding said second fluid storage structure in the thickness dimension.
2. The disposable absorbent product of claim 1, wherein said core includes an acquisition layer adjacent said first and second fluid storage structures and configured to absorb and distribute fluid secreted by the wearer toward said first and second fluid storage structures.
3. The disposable absorbent product of claim 2, wherein said acquisition layer is free of superabsorbent material.
4. The disposable absorbent product of any of claims 1 to 3, wherein said first fluid storage structure includes a top surface adjacent said topsheet, and a bottom surface adjacent said backsheet, said first fluid storage structure defining a hole extending between said top surface and said bottom surface, said second fluid storage structure being located within said hole.
5. The disposable absorbent product of any of claims 1 to 3, wherein said first fluid storage structure has an inner portion having a first thickness, and an outer portion having a second thickness greater than said first thickness and surrounding said inner portion in the thickness dimension, said second fluid storage structure being supported within said inner portion.
6. The disposable absorbent product of any of claims 1 to 5, wherein said first and second fluid storage structures include respective first and second, generally coplanar top surfaces, both adjacent said topsheet.
7. The disposable absorbent product of claim 5, wherein said inner portion has a first density and said outer portion has a second density, said first density being greater than said second density.
8. The disposable absorbent product of any of claims 1 to 7, wherein said second fluid storage is spaced from said first fluid storage structure at least along a portion of a periphery of said second fluid storage structure.
9. The disposable absorbent product of any of claims 1 to 8, wherein said first fluid storage structure comprises pulp.
10. The disposable absorbent product of any of claims 1 to 9, wherein said second fluid storage structure has a substantially rectangular periphery.
11. The disposable absorbent product of any of claims 1 to 10, wherein said second fluid storage structure comprises an airlaid material.
12. The disposable absorbent product of any of claims 1 to 11, wherein said second fluid storage structure comprises superabsorbent material.
13. The disposable absorbent product of any of claims 1 to 12, wherein said second fluid storage structure has an absorption capacity of at least about 60 grams Rothwell.
14. A method of forming a core in a disposable absorbent product that includes a topsheet and a backsheet overlaying the topsheet, the core being disposed between the topsheet and backsheet and having a length dimension, a width dimension, and a thickness dimension orthogonal to the length and width dimensions, the method comprising:

obtaining a first fluid storage structure having a top surface, a bottom surface disposed opposite the top surface, a plurality of outer walls extending between the top and bottom surfaces, and a plurality of inner walls, the outer and inner walls substantially extending along dimensions orthogonal to the thickness dimension, the inner walls defining an opening in the first fluid storage structure;

locating a second fluid storage structure within the opening; and

placing the first and second fluid storage structures between the topsheet and backsheet to thereby define the core of the disposable absorbent product.
15. The method of claim 14, wherein the inner walls extend between the top and bottom surfaces.
16. The method of claim 14, wherein the inner walls extend from the top surface to a plane intermediate between the top and bottom surfaces.
17. The method of claim 14 or 16, further comprising:

compressing an inner portion of the first fluid storage structure to a predetermined degree, while leaving an outer portion thereof surrounding the inner portion substantially uncompressed, so as to define the opening.
18. The method of claim 14 or 16, further comprising:

compressing an outer portion of the first fluid storage structure to a first extent;

compressing an inner portion of the first fluid storage structure surrounded by the inner portion to a second extent greater than the first extent, so as to define the opening.
19. The method of any of claims 14 to 18, wherein

the first and second fluid storage structures include respective first and second top surfaces adjacent the topsheet, and

locating the second fluid storage structure within the opening includes defining a gap between the first and second fluid storage structures at least along a portion of the periphery of the second fluid storage structure.
20. The method of any of claims 14 to 18, wherein

the first and second fluid storage structures include respective first and second top surfaces adjacent the topsheet, and

locating the second fluid storage structure within the opening such that the first and second top surfaces are substantially coplanar.
21. The method of claim 19, further comprising:

locating the second fluid storage structure within the opening such that the first and second top surfaces are substantially coplanar.
22. An absorbent core for use in an absorbent product, the absorbent core comprising:

a first fluid storage structure; and

a second fluid storage structure, wherein

the core has a length dimension, a width dimension, and a thickness dimension orthogonal to the length and width dimensions, and said first fluid storage structure at least partially surrounds said second fluid storage structure in the thickness dimension.

Other embodiments of an absorbent core are contemplated having the characteristics of claim 22, as described above, and further having the features described in any of claims 2-13, above. Similarly, embodiments are contemplated of methods for making a core of the type described herein and using the methods described in any of claims 14-21, above.

From the above disclosure of the general principles of the present invention and the preceding detailed description of exemplary embodiments, those skilled in the art will readily comprehend the various modifications to which this invention is susceptible. Accordingly, this invention is intended to be limited only by the scope of the following claims and equivalents thereof.

What is claimed is:

1. A disposable absorbent product comprising:
a backsheet;
a topsheet overlaying said backsheet; and
a core disposed between said backsheet and said topsheet for retaining fluid secreted by a wearer of the absorbent product, said core having a length dimension, a width dimension, and a thickness dimension orthogonal to the length and width dimensions, said core including first and second fluid storage structures, said first fluid storage structure at least partially surrounding said second fluid storage structure in the thickness dimension.

2. The disposable absorbent product of claim 1, wherein said core includes an acquisition layer adjacent said first and second fluid storage structures and configured to absorb and distribute fluid secreted by the wearer toward said first and second fluid storage structures.

3. The disposable absorbent product of claim 2, wherein said acquisition layer is free of superabsorbent material.

4. The disposable absorbent product of claim 1, wherein said first fluid storage structure includes a top surface adjacent said topsheet, and a bottom surface adjacent said backsheet, said first fluid storage structure defining a hole extending between said top surface and said bottom surface, said second fluid storage structure being located within said hole.

5. The disposable absorbent product of claim 1, wherein said first fluid storage structure has an inner portion having a first thickness, and an outer portion having a second thickness greater than said first thickness and surrounding said inner portion in the thickness dimension, said second fluid storage structure being supported within said inner portion.

6. The disposable absorbent product of claim 5, wherein said first and second fluid storage structures include respective first and second, generally coplanar top surfaces, both adjacent said topsheet.

7. The disposable absorbent product of claim 5, wherein said inner portion has a first density and said outer portion has a second density, said first density being greater than said second density.

8. The disposable absorbent product of claim 1, wherein said second fluid storage is spaced from said first fluid storage structure at least along a portion of a periphery of said second fluid storage structure.

9. The disposable absorbent product of claim 1, wherein said first fluid storage structure comprises pulp.

10. The disposable absorbent product of claim 1, wherein said second fluid storage structure has a substantially rectangular periphery.

11. The disposable absorbent product of claim 1, wherein said second fluid storage structure comprises an airlaid material.

12. The disposable absorbent product of claim 11, wherein said second fluid storage structure comprises superabsorbent material.

13. The disposable absorbent product of claim 1, wherein said second fluid storage structure has an absorption capacity of at least about 60 grams Rothwell.

14. A disposable absorbent product comprising:
a backsheet;
a topsheet overlaying said backsheet; and
a core disposed between said backsheet and said topsheet for retaining fluid secreted by a wearer of the absorbent product, said core having a length dimension, a width dimension, and a thickness dimension orthogonal to the length and width dimensions, said core including a first fluid storage structure including pulp, and a second fluid storage structure including an airlaid material, said first fluid storage structure at least partially surrounding said second fluid storage structure in the thickness dimension.

15. The disposable absorbent product of claim 14, wherein said first and second fluid storage structures include respective first and second, generally coplanar top surfaces, both adjacent said topsheet.

16. The disposable absorbent product of claim 15, wherein said first fluid storage structure includes a first bottom surface, opposite said first top surface thereof, adjacent said backsheet, said first fluid storage structure defining a hole extending between said first top surface and said first bottom surface, said second fluid storage structure being located within said hole.

17. The disposable absorbent product of claim 15, wherein said first fluid storage structure has an inner portion having a first thickness, and an outer portion having a second thickness greater than said first thickness and surrounding said inner portion in the thickness dimension, said second fluid storage structure being supported within said inner portion.

18. The disposable absorbent product of claim 14, wherein said second fluid storage structure has an absorption capacity of at least about 60 grams Rothwell.

19. An absorbent core for use in an absorbent product, the absorbent core comprising:
a first fluid storage structure; and
a second fluid storage structure, wherein
the core has a length dimension, a width dimension, and a thickness dimension orthogonal to the length and width dimensions, and
said first fluid storage structure at least partially surrounds said second fluid storage structure in the thickness dimension.

20. A method of forming a core in a disposable absorbent product that includes a topsheet and a backsheet overlaying the topsheet, the core being disposed between the topsheet and backsheet and having a length dimension, a width dimension, and a thickness dimension orthogonal to the length and width dimensions, the method comprising:
obtaining a first fluid storage structure having a top surface, a bottom surface disposed opposite the top surface, a plurality of outer walls extending between the top and bottom surfaces, and a plurality of inner walls, the outer and inner walls substantially extending along dimensions orthogonal to the thickness dimension, the inner walls defining an opening in the first fluid storage structure;
locating a second fluid storage structure within the opening; and
placing the first and second fluid storage structures between the topsheet and backsheet to thereby define the core of the disposable absorbent product.

21. The method of claim 20, wherein the inner walls extend between the top and bottom surfaces.

22. The method of claim 20, wherein the inner walls extend from the top surface to a plane intermediate between the top and bottom surfaces.

23. The method of claim 22, further comprising:
compressing an inner portion of the first fluid storage structure to a predetermined degree, while leaving an outer portion thereof surrounding the inner portion substantially uncompressed, so as to define the opening.

24. The method of claim 22, further comprising:
compressing an outer portion of the first fluid storage structure to a first extent;
compressing an inner portion of the first fluid storage structure surrounded by the inner portion to a second extent greater than the first extent, so as to define the opening.

25. The method of claim 20, wherein
the first and second fluid storage structures include respective first and second top surfaces adjacent the topsheet, and
locating the second fluid storage structure within the opening includes defining a gap between the first and second fluid storage structures at least along a portion of the periphery of the second fluid storage structure.

26. The method of claim 20, wherein the first and second fluid storage structures include respective first and second top surfaces adjacent the topsheet, the method further comprising:
locating the second fluid storage structure within the opening such that the first and second top surfaces are substantially coplanar.
